(19) United States
(12) Patent Application Publication
(10) Pub. No.: US 2018/0003579 A1
(43) Pub. Date: Jan. 4, 2018

(54) SENSORS, INTERFACES AND SENSOR SYSTEMS FOR DATA COLLECTION AND INTEGRATED MONITORING OF CONDITIONS AT OR NEAR BODY SURFACES

(71) Applicant: Sensoria Inc., Redmond, WA (US)

(72) Inventors: Mario ESPOSITO, Redmond, WA (US); Victoria ESPOSITO, Redmond, WA (US); Christopher SMALL, Renton, WA (US); Maria CARMAGNANI, Redmond, WA (US); Matthew KUEPER, Kirkland, WA (US)

(21) Appl. No.: 15/540,404

(22) PCT Filed: Dec. 30, 2015
(86) PCT No.: PCT/US2015/068180

§ 371 Date: Jun. 28, 2017

Related U.S. Application Data
(60) Provisional application No. 62/163,861, filed on May 19, 2015.
(60) Provisional application No. 62/099,099, filed on Dec. 31, 2014.

(51) Int. Cl. G01L 5/00; A61B 5/0488; A41B 9/00; A41B 9/08; A41B 11/00; A41C 3/00; A41D 1/00; A41D 1/08; A41D 19/00; A61F 13/00; A61B 5/00; A41B 1/08; A61B 5/0205; A43B 13/00; A43B 3/00; A42B 1/24; A41F 9/00; G01L 1/20; A61B 5/145; A61B 5/024; A61B 5/053; A61B 5/08; A61B 5/103; A61B 5/11

(52) U.S. Cl. CPC G01L 5/0052; G01L 1/205; A61B 5/0488; A41B 9/00; A41B 9/08; A41B 11/00; A41C 3/0064; A41D 1/002; A41D 1/08; A41D 19/0027; A61F 13/00051; A61B 5/6895; A61B 5/6807; A61B 5/6806; A61B 5/6803; A41B 1/08; A61B 5/02055; A43B 13/00; A43B 3/0005; A42B 1/242; A41F 9/00; A61B 5/14542; A61B 5/02438; A61B 5/0533; A61B 5/08; A61B 5/1038; A61B 2562/0219; A61B 5/1112; A61F 2013/00089

(57) ABSTRACT

Sensing devices including sensors such as flexible and stretchable fabric-based pressure sensors, may be associated with or incorporated in garments, such as socks, intended to be worn against a body surface (directly or indirectly). Specific manifestations of a sensing system incorporated in a sock substrate are described in detail. Dedicated electronic devices interface electrically with sensors through intermediate conductive traces, optional conductive bridges, conductive contacts provided in a mounting tab.

### REFERENCE TO RELATED APPLICATIONS

This application claims priority to U.S. Patent Application Nos. 62/099,099 and 62/163,861, filed Dec. 31, 2014 and May 19, 2015, respectively. The disclosures of these priority applications are incorporated herein by reference in their entireties.

### FIELD

The present invention relates generally to sensors, including flexible and stretchable fabric-based pressure sensors associated with or incorporated in garments and other substrates intended to contact a body surface (directly or indirectly), and to sensor interfaces with electronic components and devices. Several aspects of detailed sensor system components and integration of sensing systems in garments are disclosed.

### BACKGROUND

Various types of sensing systems have been incorporated in shoes, insoles, socks and other types of garments for monitoring various physiological parameters for various applications, including recreational, fitness, sporting, military, diagnostic and medical applications. The use of sensing systems for fitness applications to monitor and analyze activities such as running, walking, energy expenditure, and the like, is now common. Medical applications for sensing pressure, temperature and the like for purposes of monitoring neuropathic and other degenerative conditions with the goal of alerting an individual and/or medical service providers to sensed parameters that may indicate the worsening of a condition, lack of healing, and the like, have been proposed. Footwear-related sensing systems directed to providing sensory data for patients suffering from neuropathy, for gait analysis, rehabilitation assessment, shoe research, design and fitting, orthotic design and fitting, and the like, have been proposed.

Sensing devices and footwear having sensors incorporated for monitoring pressure and other body parameters have been proposed. Various types of sensing systems for monitoring various physiological parameters have been incorporated in bands, wrist-worn devices, portable electronic devices, medical devices, shoes, insoles, socks and other types of garments for various applications. The use of sensing systems for fitness applications to monitor and analyze activities such as running, walking, energy expenditure, and the like, is now common. Medical applications for sensing pressure, posture, gait, temperature and the like for purposes of monitoring neuropathic and other degenerative conditions with the goal of alerting an individual and/or medical service providers to sensed parameters that may indicate the worsening of a condition, lack of healing, and the like, have been proposed. Footwear-related sensing systems directed to providing sensory data for patients suffering from neuropathy, for gait analysis, rehabilitation assessment, shoe research, design and fitting, orthotic design and fitting, and the like, have been proposed.

In one aspect, the components and assemblies for collection and analysis of data from sites such as feet and other body surfaces described herein are directed to providing intermittent and/or continuous monitoring and reporting of conditions and activity parameters (such as force, pressure, shear, etc.) at body locations and combining that data with additional data for purposes of analyzing and reporting activity parameters and providing feedback to the user or a third party, such as a coach in fitness applications, a caretaker or medical professional in medical applications, or the like. In other aspects, the components and assemblies for collection and analysis of data from sites such as feet and other body surfaces described herein are directed to reducing the incidence and severity of gait problems and wounds, improving a user's gait, providing information to caretakers, increasing compliance with prescribed regimen, and accelerating the pace and quality of wound healing. In yet other aspects, sensors, interfaces, systems and materials described herein for collection and analysis of physiological and biomechanical data from sites such as feet and other body parts may be used for a variety of sports-related, military, fitness, medical, diagnostic and therapeutic purposes.

### SUMMARY

In one aspect, sensor systems of the present invention comprise one or more sensor(s) mounted to or incorporated in or integrated with or associated with (referred to herein, collectively, as “associated with”) a substrate material such as a wearable garment, a wearable band, an independently positionable component, or another substrate, such as a bandage or a flexible and/or pliable sheet material. In one aspect, sensors are capable of sensing a physiological parameter of the underlying skin or tissue; in some aspects, sensors are capable of sensing force and/or pressure and/or shear exerted on or against an underlying skin or tissue. In some aspects, sensors are capable of sensing conductive impulses or properties associated with a body surface or tissue. Each sensor is electrically connected, optionally via one or more flexible leads, to a flexible and conductive trace associated with the substrate, and conductive traces terminate at conductive signal transfer terminals associated with the substrate. Each sensor may additionally be connected to a ground trace terminating at a ground terminal.

Sensor systems and sensing devices described herein preferably comprise at least one flexible sensor (or, means for sensing), and one or more of the sensor(s), flexible leads, and conductive traces may be stretchable and/or elastic as well as being flexible. In some embodiments, the sensor(s), flexible leads and conductive traces may all comprise pliable, electrically resistive and/or conductive fabric materials. Garments and other types of substrates incorporating such sensor systems and sensing devices may be comfortably worn by users, and/or contact a body surface of users, under many conditions, providing real time monitoring of conditions at or near body surfaces.

The signal transfer terminal(s) on the substrate may be matingly received in signal receipt terminals associated with a Dedicated Electronic Device (DED) that is mountable to the substrate or electrically connects to the signal transfer terminal(s) and serves as a (temporary or permanent) data collection device. The DED may also (optionally) house batteries or other energy storage (and/or energy generating devices) and serve as a sensor charging device. The DED may additionally communicate with one or more external electronic device(s), such as a smartphone, personal computing device/display, host computer, or the like for signal transfer, processing, analysis and display to a user and/or others. In some embodiments, the external electronic device, and/or the DED, communicates with an external, hosted computing system (operated, e.g., at a centralized, hosted facility and/or in the “Cloud”) that provides additional data analysis, formulates feedback, notifications, alerts, and the like, that may be displayed to the user, a coach, a caretaker, a clinician, or the like, through one or more computing and/or display devices. In alternative embodiments, the DED may itself perform signal processing and analysis, and may display or otherwise communicate feedback directly to a user without interfacing with an external computing device. In some embodiments, the DED is detachably attachable to signal receipt terminals incorporated in an interface component associated with a substrate.

In some embodiments, one or more resistive sensor(s) detect changes in voltage or resistance across a surface area that is associated with force exerted on the sensor, which is related to pressure (as force per unit surface area) and/or shear. Force, pressure, shear and measurements or values that are derivative thereof may therefore be determined at identifiable spatial locations where sensors are positioned. E-textile sensors capable of providing proportional pressure signals (e.g., proportional pressure sensed over a surface area), and/or providing pressure signals that correlate with spatial locations on a surface area of the e-textile sensors are preferred for many applications.

In some embodiments, FSR (Force Sensitive Resistor) or piezo-resistive sensors may be used. One type of piezoresistive force sensor that has been used previously in footwear pressure sensing applications, known as the FLEXIFORCE® sensor, can be made in a variety of shapes and sizes, and measures resistance, which is inversely proportional to applied force. These sensors use pressure sensitive inks with silver leads terminating in pins, with the pressure sensitive area and leads sandwiched between polyester film layers. FLEXIFORCE® sensors are available from Tekscan, Inc., 307 West First Street, South Boston, Mass. 02127-1309 USA. Other types of sensors, including sensors employing conductive electrodes, may also be associated with various substrate materials (e.g., garments, sheet materials and the like). Such sensors may provide data relating to temperature, moisture, humidity, stress, strain, heart rate, respiratory rate, blood pressure, blood oxygen saturation, blood flow, local gas content, galvanic skin response, bacterial content, position, multi-axis acceleration, as well as locational positioning (GPS), and the like. A variety of such sensors are known in the art and may be adapted for use in sensing systems described herein.

In some embodiments, sensors and/or associated leads and/or conductive traces incorporated in sensing systems of the present invention comprise non-silicon-based materials such as flexible, resistive and/or conductive “e-textile” fabric and/or yarn material(s). In some embodiments, sensors and/or associated leads and/or conductive traces incorporated in sensing systems of the present invention comprise flexible, resistive and/or conductive fabric or yarn materials that are substantially isotropic with respect to their flexibility and/or stretch properties. By “substantially” isotropic, we mean to include materials that have no more than a 15% variation and, in some embodiments, no more than a 10% variation in flexibility and/or stretch properties in any direction, or along any axis of the material. Suitable materials, such as resistive and/or piezoresistive or conductive fabric and yarns, coated and/or impregnated fabrics and yarns, such as metallic coated fabric and yarn materials and fabric and yarn materials coated or impregnated with other types of resistive or conductive formulations, are known in the art and a variety of such fabric and yarn materials may be used. In some embodiments, pressure sensors comprise flexible conductive woven fabric material that is stretchable and/or elastic and/or substantially isotropic with respect to its flexibility and/or stretch properties.

Fabrics and yarns comprising a knitted nylon/spandex substrate coated with a resistive formulation are suitable for use, for example, in fabricating biometric e-textile pressure sensors and in other applications requiring environmental stability and conformability to irregular configurations. One advantage of using these types of e-textile sensors is that they perform reliably in a wide variety of environments (e.g., under different temperature and moisture conditions), and they're generally flexible, durable, washable, and comfortably worn against the skin. Suitable flexible resistive fabric and yarn materials are available, for example, from VTT/Shieldex Trading USA, 4502 Rt-31, Palmyra, N.Y. 14522, from Statex Productions & Vertriebs GmbH, Kleiner Ort 11 28357 Bremen Germany, and from Eeonyx Corp., 750 Belmont Way, Pinole, Calif. 94564.

Techniques for deriving force and/or pressure and/or shear measurements using e-textile materials are known in the art and various techniques may be suitable. See, e.g., http://www.kobakant.at/DIY/?p=913. Techniques for measuring other parameters using e-textile materials, such as humidity and temperature measurements, are also known and may be used in sensing systems of the present invention. See, e.g., http://www.nano-tera.ch/pdf/posters2012/TWIGS105.pdf. E-textile sensors of the present invention may thus be capable of monitoring various parameters, including force, pressure, shear, humidity, temperature, gas content, and the like, at the sensor site. Additional monitoring capabilities may be available using e-textile sensors as innovation in fabric sensors proceeds and as nano-materials and materials incorporating nano-structures are developed and become commercially feasible.

Flexible (and optionally stretchable or elastic) resistive and/or conductive fabric sensor(s), leads and/or traces may be associated with an underlying substrate such as fabric or sheet material that's substantially non-conductive and flexible. The term “fabric” or “sheet material” as used herein, refers to many types of pliable materials, including traditional fabrics comprising woven or non-woven fibers or strands, knitted substrates and materials, as well as fiber reinforced sheet materials, and other types of flexible sheeting materials composed of natural and/or synthetic materials, including flexible plastic sheeting material, pliable thermoplastic, foam and composite materials, screen-like or mesh materials, and the like. The underlying substrate may comprise a sheet material fabricated from flexible fabric material that is stretchy and/or elastic. The sheet material forming the underlying substrate may be substantially isotropic with respect to its flexibility and/or stretch properties. By “substantially” isotropic, we mean to include materials that have no more than a 15% variation and, in some embodiments, no more than a 10% variation in flexibility and/or stretch properties in any direction, or along any axis of the material.

Flexible resistive or conductive yarns may also be used to fabricate e-textile sensors. Such resistive or conductive yarns may be woven or knit or otherwise associated with or integrated in a substrate material according to predetermined patterns to provide a plurality of spatially distinctive sensors associated with a substrate such as a garment. In one embodiment, garments having a plurality of integrated and spatially distinct resistive or conductive sensors fabricated by weaving or knitting or otherwise associating resistive or conductive yarns or fibers in substrate materials are preferred.

For garment and similar applications, for example, one or more e-textile sensor(s) and/or sensing devices may be associated with (e.g., sewn or otherwise permanently or detachably attached or connected or fixed to, or woven or knit or integrated in) a garment or a surface of a garment for contacting an individual's skin, directly or indirectly, during use. Conductive sensors may be used, for example, to detect electrical impulses for monitoring vital signs, skin conductance, or the like; resistive sensors may be used, for example, for detecting pressure and/or force and/or shear exerted against an individual's skin or other force-related parameters sensed at or near a skin surface.

In situations where parameters are desired to be measured as they impact an outer surface or fabric layer, one or more sensor(s) may be associated with (e.g., mounted to or woven or knit or integrated in) an external surface of a garment or substrate. For applications such as bands, bandages and independently positionable sensing components, e-textile sensors may likewise be mounted to or otherwise associated with an underlying substrate that may be conveniently positioned as desired by the user or a third party, such as a caretaker or clinician. In alternative embodiments, e-textile sensors may be sandwiched between substrate layers (as in compression socks or other types of compression garments and substrates) or otherwise incorporated in various types of substrates.

Each sensor may be associated with one or more leads, each of the leads being electrically connected to a conductive trace conveying electrical signals from the sensor to a signal transfer terminal. In some embodiments, e-textile sensors as previously described may be electrically connected to lead(s), or e-textile sensors may have flexible yarn or textile lead(s) associated with or incorporated in the e-textile sensor footprint. The lead(s) are electrically connected to flexible conductive traces, which may comprise a variety of flexible conductive materials, such as a conductive fabric, yarn, fibers or the like. In some embodiments, the conductive traces are stretchable and/or elastic, and are woven or knit into and form part of the substrate. In some embodiments, the conductive traces are insulated or have an insulative coating.

In some embodiments, conductive traces comprise a conductive e-textile fabric having generally high electrical conductivity, such as silver coated e-textile materials, and may be bonded to the underlying substrate material using adhesives, heat bonding or non-conductive threads. Suitable e-textile materials are known in the art and are available, for example, from the vendors identified above. In some embodiments, conductive traces comprise a conductive yarn or fiber having generally high electrical conductivity, and the yarn or fiber materials are integrated into the substrate material by knitting, weaving, or the like. In some embodiments, the conductive traces comprise a conductive yarn or fiber having generally high electrical conductivity, and having an insulative coating, and the insulated, conductive yarn or fiber materials are integrated into the substrate material by knitting, weaving, or the like. In some embodiments, conductive traces comprise other types of flexible conductive materials, such as thermoplastic elastomers (TPEs), conductive inks, or the like. Conductive traces comprising materials such as TPEs, conductive inks, and the like, may be associated (directly or indirectly) with a substrate and with one or more leads or sensors to provide conductive pathways between the sensors and corresponding signal transfer terminals.

Sensor(s) as described herein and sensor systems, including e-textile pressure sensors and a variety of other types of sensors, with (optional) leads and conductive traces, may be associated with a variety of substrates including, without limitation, garments intended to be worn (directly or indirectly) against the skin of an individual, such as shirts, tunics, shorts, body suits, leotards, underwear, leggings, socks, footies, gloves, caps, bands such as wrist bands, leg bands, torso and back bands, brassieres, and the like. Sensors and sensor systems may additionally be associated with wraps having different sizes and configurations for fitting onto or wrapping around a portion of an individual's body, and with bands, bandages, and wound dressing materials, as well as with other types of accessories that contact a user's body surface (directly or indirectly) such as insoles, shoes, boots, belts, straps, and the like.

Each of the conductive traces terminates in a signal transfer terminal that is mounted to/in/on, or otherwise associated with (referred to, collectively, as “associated with”), the underlying substrate and can be contacted to a mating signal receipt terminal of a dedicated electronic device (DED) having data storage, processing and/or analysis capabilities. In general, conductive traces and terminals are arranged in a predetermined arrangement that corresponds to the arrangement of signal receipt terminals in the DED. Many different types of signal transfer and receipt terminals are known and may be used in this application. In one exemplary embodiment, signal transfer and receipt terminals may be mounted in cooperating fixtures for sliding engagement of the fixtures and terminals. In another embodiment, signal transfer terminals may be provided as conductive fixtures that are electrically connected to the conductive trace (and thereby to a corresponding sensor) and detachably connectible to a mating conductive fixture located on the DED.

In some embodiments, the mating signal receipt and signal transfer terminals may comprise mechanically mating, electrically conductive members such as snaps or other types of fasteners providing secure mechanical mating and high integrity, high reliability transfer of signals and/or data. In some embodiments, the mating terminals may comprise conductive pins, including stationary conductive pins as well as movable pins, such as spring-loaded pins, referred to as pogo pin connectors. In some embodiments, easy and secure mating of the terminals may be enhanced using magnetic mechanisms or other types of mechanisms that help users to properly and securely align and connect/disconnect the mating terminals with minimal effort. In some embodiments, easy and secure mating of the terminals may be enhanced by complementary (and/or locking) mechanical configurations of housing components associated with mating terminals. In some embodiments, mating terminals provided on the underlying substrate (e.g., a garment, sock, sheet, band, etc.) and on a DED are provided in a predetermined arrangement, or have a keyed configuration, to ensure that the DED is properly aligned and mounted to the terminals provided on the substrate in a predictable and pre-determined orientation.

The DED, in addition to having data recording, processing and/or analysis capabilities, may incorporate an energy source such as a battery providing energy for data recording, processing and/or analysis, as well as providing energy for operation of one or more of the sensor(s). The energy source may comprise a rechargeable and/or replaceable battery source, and/or a regenerative energy system. The DED generally provides a lightweight and water-tight enclosure for the data collection and processing electronics and (optional) energy source and provides signal receiving terminals that mate with the signal transfer terminals connected to the sensor(s) for conveying data from the sensors to the dedicated electronic device. In some embodiments, the DED is provided as a bendable or partially bendable device that can be shaped, as desired, to fit comfortably on and closely to body surfaces having different configurations and sizes.

A DED may be provided in the form of a curved band for mounting to the user at or near the user's ankle, and particularly at or near a front-facing portion of the user's ankle, for example, and may be at least partially flexible so that it fits, comfortably and functionally, on men's and women's ankles and on ankles having different sizes and shapes, providing connection to the sensor transfer terminals provided in a sock or anklet. In some embodiments, a partially or fully bendable DED may be used in a variety of configurations, including, e.g., flat or substantially flat configurations, depending on the location of sensor transfer terminals provided in an underlying substrate. In some embodiments, a partially or fully bendable DED may be used in different configurations with sensor transfer terminals provided in different form factors. For example, a common DED may be shaped to fit comfortably on a user's ankle and mate with sensor transfer terminals provided on an underlying sock or anklet; it may also be shaped to fit comfortably on a user's arm and/or wrist and mate with sensor transfer terminals provided on an underlying sleeve. The same bendable DED may additionally be shaped to fit comfortably, in a generally curved or a generally flat configuration, and mate with sensor transfer terminals provided on garments or substrates having other form factors.

DEDs having alternative configurations are also disclosed and may be used in a variety of applications. In some embodiments, a DED may be provided in the form of a button-like or dongle-like or capsule-like object having signal receipt terminals that mate with signal transfer terminals provided in a mating DED-receiving fixture that may be mounted to or incorporated in (referred to, collectively, as “associated with”) an underlying surface of a garment or another substrate. In some embodiments, the DED-receiving fixture may comprise a substantially flexible and bendable material and may be mounted to a sock substrate at or near a user's ankle In some embodiments, the DED-receiving fixture may be associated with an underlying garment at different garment regions, and multiple DED-receiving fixtures and DEDs may be used for various monitoring and data collection purposes.

When sensors are incorporated in a shirt-like garment or tunic and signal transfer terminals are arranged on a front or back surface of the garment, the DED may have a generally medallion-like form factor, or a button-like or linear or another form factor, depending on the placement and type of signal transfer terminals, the underlying conformation of the body surface, and the like. When sensors are incorporated in a wrap or band or sheet-like substrate, the signal transfer terminals may be arranged at or near an exposed end of the wrap or band or sheet following its application to an underlying anatomical structure or body surface or substrate, and the DED may be provided as a band or a tab or a button-like or dongle-like or capsule-like device having aligned signal receipt terminals. The DED may be provided as a substantially flexible or a substantially rigid component, depending upon the application, and it may take a variety of forms.

In some embodiments, the DED communicates with and transfers data to one or more external computing and/or display system(s), such as a smartphone, computer, tablet computer, dedicated computing device, medical records system or the like, using wired and/or wireless data communication means and protocols. The DED and/or an external computing and/or display system may, in turn, communicate with a centralized host computing system (located, e.g., in the Cloud), where further data processing and analysis takes place. Substantially real-time feedback, including data displays, notifications, alerts and the like, may be provided to the user, caretaker and/or clinician according to user, caretaker and/or clinician preferences.

In some embodiments, the DED may store data temporarily to a local memory, and may periodically transfer the data (e.g., in batches) to the above mentioned external computing and/or display system(s). Offline processing and feedback, including data displays, notifications and the like may be provided to the user, caretaker, and/or clinician according to user, caretaker and/or clinician preferences.

In operation, an authentication routine and/or user identification system matches the DED and associated sensing system (e.g., the collection of sensor(s) associated with an underlying substrate) with the user, caretaker and/or clinician, and may link user information or data from other sources to a software- and/or firmware-implemented system residing on the external computing system. The external computing device may itself communicate with a centralized host computing system or facility where data is stored, processed, analyzed, and the like, and where output, communications, instructions, commands, and the like may be formulated for delivery back to the user, caretaker and/or clinician through the external computing device and/or the DED.

Calibration routines may be provided to ensure that the DED and connected related sensor system are properly configured to work optimally for the specific user. Configuration and setup routines may be provided to guide the user (or caretaker or medical professional) to input user information or data to facilitate data collection, and various protocols, routines, data analysis and/or display characteristics, and the like, may be selected by the user (or caretaker or medical professional) to provide data collection and analysis that is targeted to specific users. Specific examples are provided below. Notification and alarm systems may be provided, and selectively enabled, to provide messages, warnings, alarms, and the like to the user, and/or to caretakers and/or medical providers, substantially in real-time, based on sensed data.

Various other aspects of sensing systems and background relating to the construction, use and utility for such sensing systems are described in the following previously published and commonly owned patent publications, all of which are incorporated herein by reference in their entireties: U.S. Pat. No. 8,925,392; PCT Patent Publication 2013/116242 A2; PCT Patent Publication 2015/017712 A1; U.S. Patent Publication US-2015-0182843-A1; and PCT Patent Publication WO 2015/175838 A1.

### BRIEF DESCRIPTION OF THE DRAWINGS

FIGS. 1 and 2 show images illustrating plan views of a pair of socks (right and left indicated on the cuff in FIG. 2) incorporating a sensing system as described herein. FIG. 1 shows an image illustrating a plantar view of a pair of socks incorporating a sensing system as described herein (with the left sock on the left-hand side and the right sock on the right-hand side). FIG. 2 shows an image illustrating a dorsal view of the socks illustrated in FIG. 1.

FIG. 3 shows an image illustrating a contrasting weave/knit pattern provided on the bottom of a sock and providing placement and locational positioning guidance for mounting of (discrete) sensors on the substrate.

FIG. 4A shows an image illustrating a weave/knit pattern providing placement/locational positioning guidance for conductive bridges provided at or near terminal ends of conductive traces; FIG. 4B shows an image illustrating conductive “bridges” provided at or near the terminal ends of traces for contacting data transfer terminals.

FIGS. 5A and 5B illustrate desirable distance separations between certain electrical components of a sensing system incorporated in a sock as described herein; FIG. 5C illustrates desirable contact terminal placements in proximity to terminal portions of conductive traces.

FIG. 6A shows a view illustrating contact surfaces of signal transfer terminals mounted on an inner tab surface that may be (permanently or temporarily) mounted on a substrate (e.g., a sock) with the illustrated contact surfaces arranged to contact trace terminations or conductive bridges located on the underlying substrate. FIG. 6B shows a view illustrating the tab with signal transfer terminals projecting from the outer tab surface for communicating signals from the sock sensing system to mating contacts of a DED.

FIG. 7 illustrates one embodiment of a tab having signal transfer terminals mounted in a predetermined configuration and a DED having mating signal receipt terminals.

FIGS. 8A-8F show diagrams illustrating the configuration of an exemplary tab having conductive bridges positioned on the tab at terminal locations for facilitating electrical connection between conductive terminals and traces. FIG. 8A shows a top view of one embodiment of a tab; FIGS. 8B and 8C show top perspective views of the tab of FIG. 8A; FIG. 8D shows a cross-sectional view of the tab of FIG. 8A along line A-A; FIG. 8E shows a top perspective view of conductive fittings incorporating conductive bridges according to one embodiment; and FIG. 8F shows positioning of the conductive fittings on the bottom of the tab of FIGS. 8A-8D.

FIG. 9 shows an image illustrating an enlarged view of another embodiment of an exemplary tab with conductive bridges provided on the tab for facilitating electrical connection between conductive terminals and traces.

FIGS. 10A-10C illustrate another embodiment of an exemplary tab having signal receipt terminals arranged on a tab portion and signal transfer terminals arranged in a DED receipt cavity. FIG. 10A shows an upper perspective view of the exemplary tab having a tab portion and a DED receiving fixture; FIG. 10B shows a lower perspective view of the exemplary tab illustrating signal receipt terminals provided on the bottom of the tab portion; and FIG. 10C shows a perspective view, with portions of the structure shown as transparent, illustrating the signal receipt terminals provided on the lower surface of the tab portion and signal transfer terminals provided in the DED receiving fixture. In the embodiments illustrated, the tab portion is symmetrically formed, with the right and left sides having the same appearance; likewise, both the inner and outer conformations of the DED receiving fixture are symmetrically formed, with right and left sides having the same appearance.

FIGS. 11A and 11B illustrate another embodiment of a button-like DED sized and configured for receipt in a DED receipt cavity of a tab as illustrated in FIGS. 10A-10C. FIG. 11A shows an upper perspective view of the DED, showing the external surface with an underlying interface portion, and FIG. 11B shows a lower perspective view of the DED, showing the interface portion having a plurality of signal receipt terminals extending from an internally-directed surface. In the embodiments illustrated, the DED is symmetrically formed, with right and left sides having the same appearance.

FIGS. 12A-12D illustrate a DED as illustrated in FIGS. 11A and 11B mounted in the DED receipt cavity of the exemplary tab of FIGS. 10A-10C. FIGS. 12A and 12B show plan and perspective views of the external configurations of the interfacing DED and tab components; FIG. 12C shows a top perspective view of the DED and tab components, with portions of the structure shown as transparent, illustrating the location of signal receipt terminals located on a bottom surface of the tab; and FIG. 12D shows a side view of the DED and tab components illustrating the location of signal receipt terminals on a bottom surface of the tab and signal transfer terminals located in a DED receipt cavity of the tab. In the embodiments illustrated in FIGS. 12A-12D, the external configurations of the interfacing DED and tab components are symmetrically formed, with right- and left-sides having the same configurations.

FIGS. 13A and 13B show highly schematic views of the DED and tab combination illustrated in FIGS. 10A-12D associated with a sock substrate.

FIGS. 14A-14C illustrate top perspective, bottom perspective and upper side perspective views, respectively of a charging station for a DED as illustrated in FIGS. 11A and 11B. FIG. 14A shows an upper perspective view of the external configuration of a charging station having a DED receipt cavity for mating with a DED as illustrated in FIGS. 11A and 11B. FIG. 14B shows a lower perspective view of the external configuration of a charging station of FIG. 14A. FIG. 14C illustrates an upper perspective view of the external configuration of a charging station as shown in FIG. 14A, illustrating charging pins in electrical communication with a charging interface. In the embodiment illustrated in FIGS. 14A-14C, the external configuration of the charging station is symmetrically formed, with right- and left-hand sides having the same configuration.

It will be understood that the appended drawings are not necessarily to scale, and that they present one embodiment of many aspects of systems and components of the present invention. Specific design features, including dimensions, orientations, locations and configurations of various illustrated components may be modified, for example, for use in various intended applications and environments.

### DETAILED DESCRIPTION OF EXEMPLARY EMBODIMENTS

In one embodiment, systems incorporating sensors, traces and terminals may be associated with a garment having a sock-like form factor. Although a specific embodiment of sensing systems is illustrated and described with reference to specific types of sensors, traces, bands, conductive bridges and terminals associated with a substrate having a sock-like form factor, it will be appreciated that similar fabrication techniques and features may be used in connection with a variety of sensors, traces, terminals and substrates, including other types of garments (e.g., shirts, underwear, body suits, leotards, leggings, footies, gloves, caps, sleeves, body bands and brassieres), insoles, shoes, boots, belts, straps, bandages, wraps, wrapping bands, wound dressings, sheets, pads, cushions, sporting equipment, and the like. The term “sensor,” as we use it herein, refers to the various types of sensors as described herein, as well as additional means for sensing as that term may be construed to extend to sensors as described herein as well as other, additional types of sensors that may be associated with sensing systems as described.

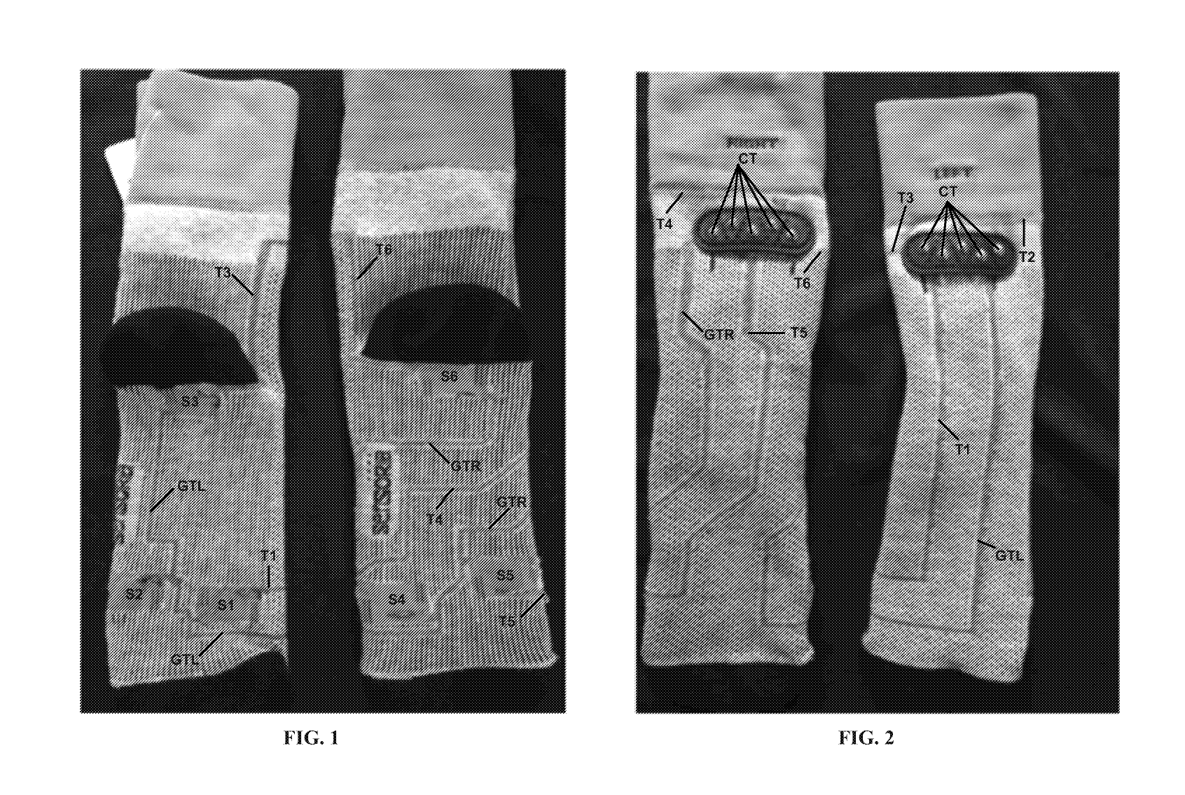

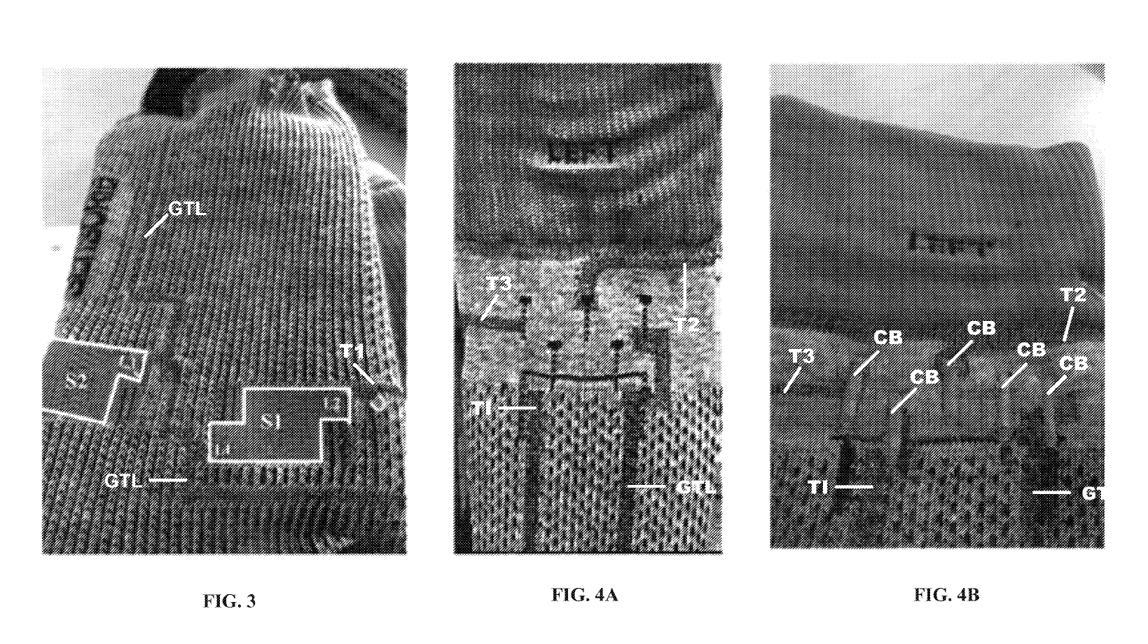

FIGS. 1 and 2 illustrate substrates in the form of socks comprising a knitted, flexible, stretchable material equipped with a plurality of sensors, leads, traces and connectors that provide signals and/or data to a dedicated (and preferably detachable) electronic device that gathers data from each sensor and communicates with an external computer and/or mobile device. In this exemplary embodiment, sensors used in footwear and sock applications may comprise e-textile pressure sensors capable of detecting levels of pressure (and/or force and/or shear or derivative measurements) at one or more areas of the foot and may include other types of sensors, including electrically conductive electrodes, vital sign monitoring sensors, accelerometers, gyroscopes, electromyography sensors, moisture sensors, and the like.

For the illustrated embodiments incorporating pressure sensors, parameters such as pressure, force and/or shear are detected at one or more areas of the foot, and trends in those parameters detected over one or more monitoring period(s) may produce conclusions relating to the user's gait, walking or running style, cadence, foot landing, susceptibility to injury, etc. can be drawn and feedback can be provided to the user, and/or to a third party (e.g., coach, care provider, physical therapist, group, etc.) to report activity, progression, susceptibility to injury, or the like. For medical applications, conclusions relating to gait, the lack of proper offloading and related conditions of the underlying skin or tissue, healing progression (or lack of healing), discomfort, extent and seriousness of injury, and the like, may be drawn and may be communicated to the user, caretaker and/or clinician, essentially in real time. In addition, notifications, alerts, recommended actions, and the like may also be communicated to the user, caretaker and/or clinician based on the data analysis, essentially in real time. These systems are suitable for use in many different types of applications.

FIG. 2 shows the exterior, dorsal (i.e., upwardly-facing when worn) surfaces of exemplary right and left socks incorporating sensing systems as described herein, with the right and left sock identifiers shown on a sock cuff. FIG. 1 shows the outside, plantar (i.e., downwardly-facing when worn) surfaces of the socks shown in FIG. 2, with the left sock on the left-hand side and the right sock on the right-hand side. The toe regions and heel regions of the socks are knit in a contrasting, black color and conductive sensor traces are visible as contrasting lines extending from and between the sensors and along the dorsal surface of the socks to signal terminals. The exterior plantar surfaces of the socks (FIG. 1) show the placement of sensors S1, S2, S3, S4, S5 and S6 (described below). In the embodiment illustrated, pressure sensor locations are located at contrasting boxes S1-S6, with two pressure sensor locations on each sock being located under the forefoot area (S1, S2, S4, S5) and one pressure sensor location on each sock being located under the heel (S3, S6). In one embodiment described in more detail below, e-textile fabric pressure sensors are associated with (e.g., fastened to, sewn to, adhered to, knit or woven into, or the like) an internal or intermediate surface of the sock at the designated sensor location(s) for contacting a user's foot (directly or indirectly) when the sock is worn. It will be appreciated that sensor placement may vary, that different types of sensors may be implemented, that additional sensors may be incorporated in sensing systems (e.g., socks) as described herein.

The material forming the substrate material of the sock is generally pliable and stretchable, and it is substantially non-electrically conductive. Natural and synthetic materials that are known and used in fabricating socks and other garments are suitable. The contrasting lines and pathways shown in FIGS. 1 and 2 leading from sensors locations S1-S6 are electrically conductive traces fabricated from electrically conductive yarns, thread, material, or the like, which may be applied or fastened or adhered to the substrate, or knit or woven into the substrate, as shown. In some embodiments, the electrically conductive traces may include an insulative cover or outer layer to provide more stable electrical signal pathways.

The conductive traces provide an electrical pathway connecting sensors or sensor leads to signal transfer terminals, which are illustrated as conductive terminals penetrating a mounting tab provided on each sock (See, FIG. 2). Traces T1, T2, T3, T4, T5 and T6 provide electrical pathways between sensors (and/or sensor leads) located at sensor locations S1-S6, respectively, and respective signal transfer terminals and extend along both plantar and dorsal regions of the sock substrate. Ground traces GTR (right sock) and GTL (left sock) extend along both plantar and dorsal regions of the sock substrate and provide electrical ground pathways contacting each sensor or sensor lead of each sock at locations S1-S3 and S4-S6, serially, and terminate at a corresponding signal transfer terminal(s). Conductive signal transfer terminals (CT) are located at trace termination locations. In the illustrated embodiment, conductive signal transfer terminals (CT) and mounting tabs are positioned in the forward-facing area of the ankle or lower leg of the user when the socks are worn. The conductive signal transfer terminals CT are illustrated mounted to the underlying sock through a flexible intermediate band component, which is described in greater detail below.

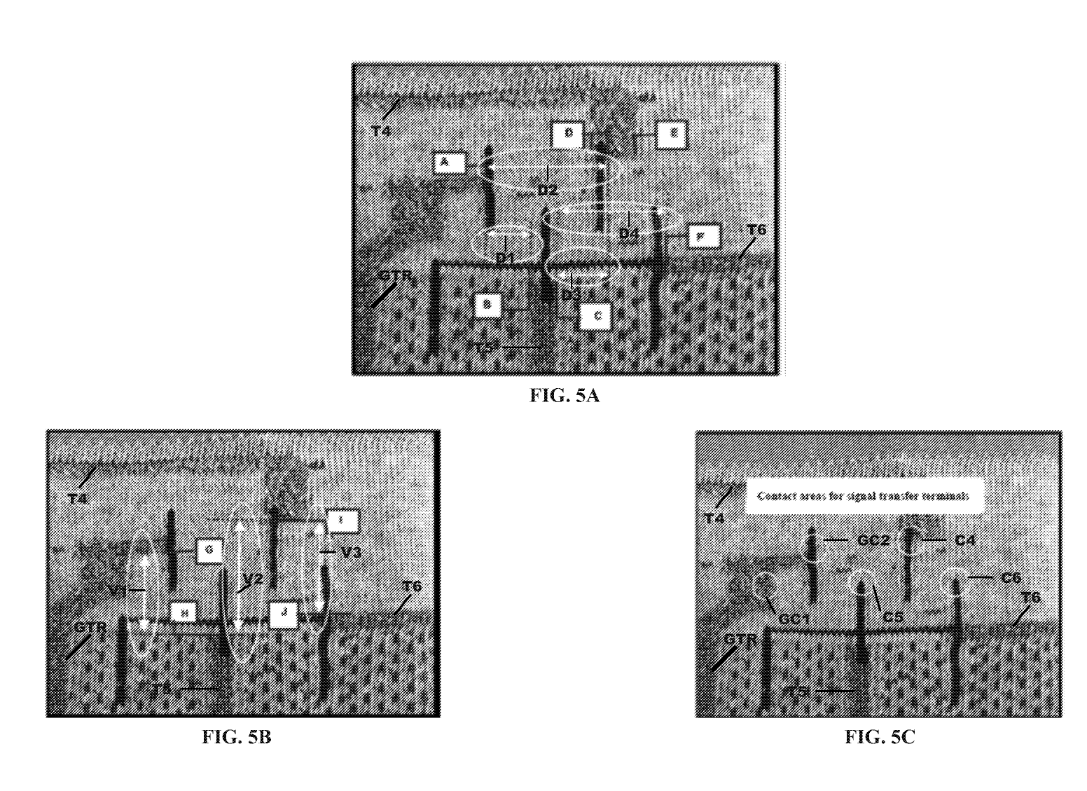

FIG. 3 illustrates an enlarged view of the exterior of a portion of the plantar surface of a sock as illustrated in FIG. 1 in a forefoot area. Two regions of a contrasting knit pattern and/or color are visible, each comprising a generally rectangular sensor location area S1, S2 of contrasting stitching with contrasting leg regions L1, L2, L3 visible extending from opposite corners of the contrasting rectangular areas S1, S2. In one aspect, this disclosure provides methods and systems for coding knit and woven substrates, including knit and woven garments, using thread, yarn or other fibers having contrasting colors, textures, materials, or the like, to indicate the location of sensors or other subsequently added components or elements to be incorporated in or associated with the garment. In this embodiment of the sensing sock illustrated in FIG. 3, pressure sensors and conductive leads are associated with an inner or intermediate surface of the sock at the corresponding regions of contrasting knit pattern. The sensor/lead placement coded by the contrasting knit pattern shown in FIG. 3 illustrates positioning of sensors S1, S2 having leads L1, L2, L3 extending from a body of the sensors S1, S2, respectively, in areas demarcated by the contrasting knit pattern and/or color. The use of contrasting color to code a substrate garment to indicate sensor location and placement reduces sensor application installation time and provides reliable sensor placement. Contrasting knit and weave patterns and colors may be conveniently programmed during weaving or knitting of the substrate using programmable weaving and knitting machinery.

The trace lines (sensor traces T1-T6 and ground traces GTR, GTL) shown in FIGS. 1 and 2 are electrically conductive traces comprising electrically conductive yarn or (elastic) thread or other material providing an electrically conductive pathway between each of the sensors or sensor leads and a location in proximity to signal transfer terminals. Electrically conductive yarns available under the mark X-STATIC are suitable for fabricating conductive traces as described herein, and insulated conductive yarns may be employed. In the embodiments illustrated, the traces (T1-T6, GTR, GTL) are integrated with the substrate material during the knitting process. This may be accomplished using programmed and/or programmable weaving or knitting machines.

Trace patterns for the left and right socks shown in FIGS. 1 and 2 are different, at least in part because the arrangement of signal transfer terminals associated with the right and left socks illustrated in FIGS. 1 and 2 is different. In some embodiments, trace patterns and the arrangement of signal transfer terminals for left and right socks may be similar or identical, or sensors, traces and signal transfer terminals on right and left socks may be provided in a mirror image arrangement. The length and width of conductive trace pathways may be modified and adjusted for different applications, depending on the impedance properties of the sensing system and electronics. For knit substrates and garments, conductive yarn or thread trace pathways are generally at least two stitches wide, and for some embodiments may be at least three stitches wide. In many embodiments, the width of one or more trace pathways formed by the conductive yarn or thread may vary over the length of the trace pathway. Thus, in some embodiments, the width of the trace pathway may vary from at least two stitches wide to three or more stitches wide. In some embodiments, wider trace pathway portions may be provided nearer the signal transfer terminal(s) and narrower trace pathway portions may be provided nearer the sensors or sensor leads.

In the embodiment illustrated in FIGS. 1 and 2, there are 3 signal transfer traces provided on each sock (one communicating with each sensor location, S1-S3, S4-S6) and one common or ground trace communicating with each sensor, in series (GTR, GTL). Each of the three signal transfer traces terminates for connection to one of the conductive signal transfer terminals. The single common or ground trace (GTR, GTL), in the embodiment shown, terminates for connection to two of the conductive terminals. In this embodiment, one of the ground terminals functions as a ground, while the other ground terminal may be provided for accomplishing functions such as sensing mating or detachment of the terminal with an associated DED and triggering activation or deactivation of the DED.

In the embodiment illustrated in FIGS. 1 and 2, the traces terminate at a front-facing, ankle region of the sock. It will be appreciated that trace termination and conductive terminal placement may be provided at a range of locations on different types of apparel and on other types of substrates. The instrumented socks shown in FIG. 2 have exposed, conductive terminals (CT) illustrated as conical, metallic components mounted through non-conductive, flexible terminal bands mounted to the sock in the area of the front, ankle region of the sock. These components, and others, are described in greater detail below.

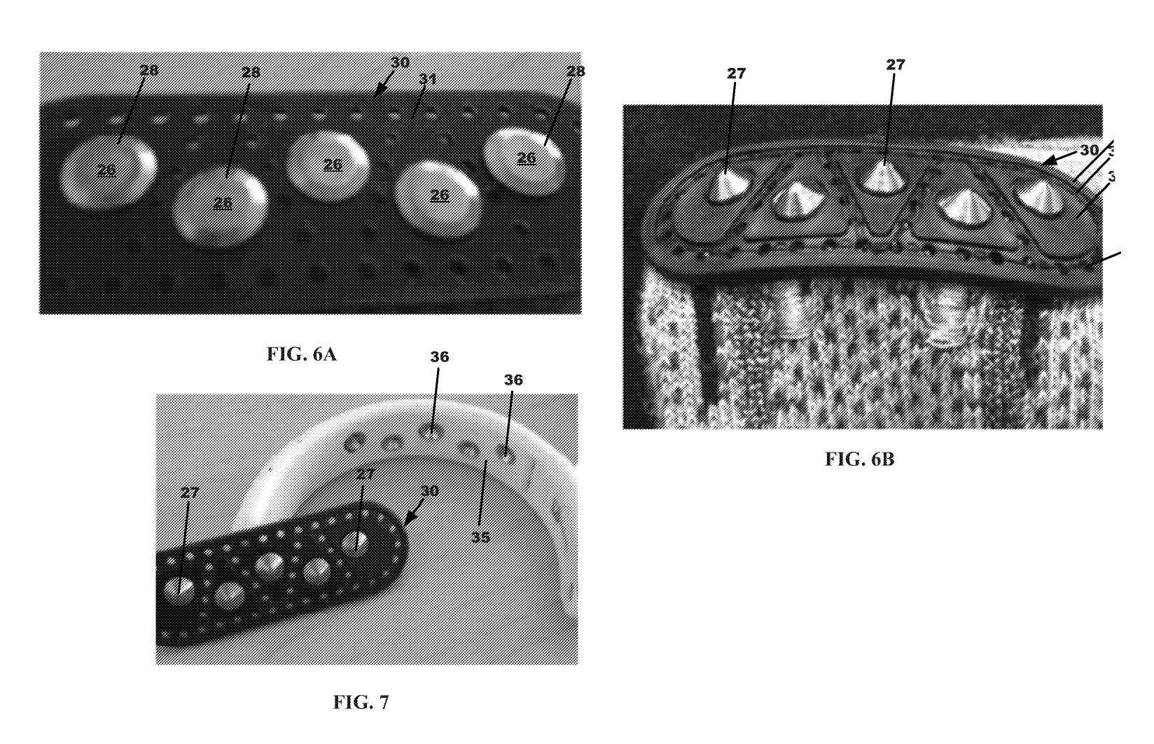

The area of trace termination is shown more clearly in FIGS. 4A and 4B. Each trace, including sensor traces (T1, T2, T3) and ground traces (GT1, GT2) terminate at an area spaced from the terminal areas of other traces. In this embodiment, conductive bridges CB, shown in FIG. 4B as longitudinally oriented areas of densely stitched conductive thread, are provided to facilitate electrical contact between a terminal area of each trace and a corresponding conductive terminal. Conductive bridges CB provide highly conductive contact areas that may be elevated with respect to the surface of the underlying substrate to facilitate stable and reliable electrical contact. In some embodiments, as shown, the conductive bridges CB are associated with the substrate and are stiffer, or less flexible, than the underlying substrate material and the flexible traces. The desired location of conductive bridges may be coded using contrasting knit patterns in the underlying substrate material, illustrated as black stitching in FIG. 4A. In some embodiments, a conductive bridge may be provided corresponding to each terminal area of each trace; in some embodiments, conductive bridges are provided for more or fewer than each of the terminal areas of each trace. Conductive bridges having configurations other than rectangular and orientations other than longitudinally directed may be provided. In some embodiments, conductive bridges provided at different trace termination locations may have different configurations, orientations, and the like.

The conductive bridges may be arranged in a staggered, offset arrangement as shown in FIG. 4B and, in some embodiments, each conductive bridge is separated from neighboring bridges by a distance of at least about 5 mm and, in some embodiments, by a distance of about 1 cm. In some embodiments, conductive bridges are separated from neighboring bridges by a distance of no less than 7 mm and no more than 1.5 cm and, in some embodiments, by a distance of no less than 7 mm and no more than 1.2 cm. The conductive threads forming conductive bridges illustrated in FIG. 4B are densely stitched in an orientation substantially transverse to the major longitudinal dimension of the formed bridge. As a result of the dense pattern of conductive material, the conductive bridges provide a substantially continuous, electrically conductive surface layer that is raised relative to the surface of the knit substrate in that area. The bridges provide a contact surface or “pad” providing a consistent and stable conductive surface for electrically contacting a “rear” face of conductive terminals. In alternative embodiments, conductive bridges comprise other types of conductive materials associated with the substrate, or with a terminal mounting band. Conductive bridges may be formed, for example, using conductive thermopolymer elastomers (TPEs), conductive inks, and the like.

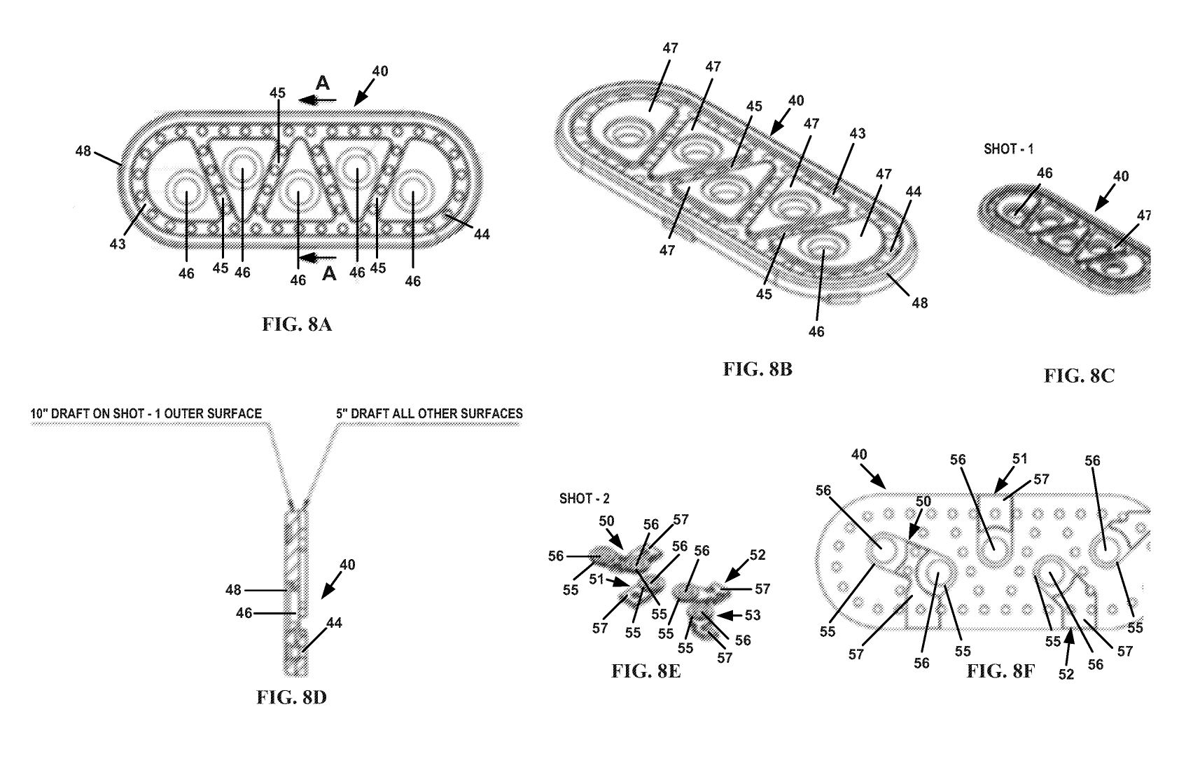

FIGS. 5A-5C illustrate another aspect of sensing systems incorporated in underlying sock substrates as described herein (and applicable to other types of substrates and substrate garments). Positioning of terminal areas of traces and conductive bridges is illustrated in FIGS. 5A and 5B, and the signal transfer terminal contact areas are shown in the super-imposed white circles of FIG. 5C. The location of trace terminal areas and signal transfer terminal contact areas shown in FIGS. 5A-5C corresponds to those of a right-hand sock as shown in FIGS. 1 and 2. Correct positioning of signal transfer terminal contact areas and conductive bridges is important to provide continuous and reliable signal transfer. Improper positioning and/or orientation may result in electrical shorts, unreliable connections, and the like. Here again, contrasting (e.g., black) stitching provided in the underlying sock substrate may be used as a guide for application of conductive bridges and/or for positioning of signal transfer terminals.

The conductive traces in this example include three sensor traces T4-T6, each communicating with a corresponding sensor (and/or sensor lead), and one common or ground trace GTR, which communicates (serially in the embodiment illustrated) with a common or ground lead of each of the sensors forming the sensing system. The common or ground trace GTR is the left-most trace shown in FIGS. 5A-5C, terminating in an enlarged conductive area having a narrow extension projecting from an upper, right-hand corner. The two left-most signal transfer terminal areas GT, GT are associated with the ground trace in this embodiment, while signal transfer terminal areas G5, G4, G6 are associated with sensor traces T5, T4, T6, respectively, as shown in FIG. 5C.

In some embodiments, as shown in FIGS. 4A and 4B, neighboring trace terminations and the associated conductive bridges are arranged in an offset, staggered arrangement. Providing sufficient horizontal and vertical spacing between edges of various traces is important; suitable positioning of trace terminations according to one embodiment is shown in FIGS. 5A and 5B. Three sensor signal traces T6, T4, T5 terminate at the three right-hand terminal locations and the ground trace GTR terminates at the two left-hand terminal locations shown in FIGS. 5A, 5B and 5C. Each of the signal traces terminates at a location (on a vertical axis) generally equidistant from each of the other signal traces. In general, each signal trace edge is separated (horizontally) a distance of at least about 4 stitches from an edge of its nearest neighboring signal trace; in some embodiments, each signal trace edge is separated (horizontally) a distance of at least about 6 stitches from an edge of its nearest neighboring trace. The ground/common trace terminates at an outside location and in some embodiments, as shown in FIGS. 5A-5C, at a location intermediate the (vertical) termination areas of the sensor traces.

The horizontal distance D1 between a terminal end of the common or ground trace GTR and the closest edge of a terminal end of sensor trace T5 (shown at B in FIG. 5A) is generally at least about 4 stitches and, in some embodiments, at least about 6 stitches. The horizontal distance D2 between a terminal end of the common or ground trace (shown at A in FIG. 5A) and the closest edge of a terminal end of sensor trace T4 (shown at D in FIG. 5A) is generally at least about 13 stitches and, in some embodiments, at least about 17 stitches. The horizontal distance D3 between the closest edge of a terminal end of sensor trace T5 (shown at C in FIG. 5A) and the closest edge of a terminal end of sensor trace T4 (shown as D in FIG. 5A) is generally at least about 5 stitches and, in some embodiments, at least about 8 stitches. The horizontal distance D4 between the closest edge of a terminal end of sensor trace T5 (shown as C in FIG. 5A) and the closest edge of a terminal end of sensor trace T6 (shown at F in FIG. 5A) is generally at least about 9 stitches and, in some embodiments, at least about 14 stitches.

In general, each signal trace edge is separated (vertically) a distance of at least about 12 stitches from an edge of its nearest neighboring trace; in some embodiments, each signal trace edge is separated (vertically) a distance of at least about 15 stitches from an edge of its nearest neighboring trace. In the illustrated embodiment, the vertical distance V1 between the lower edge of a terminal end of the ground trace (shown at G in FIG. 5B) and the upper edge of a terminal end of nearest sensor trace T5 (shown at H in FIG. 5) is generally at least about 17 stitches; the vertical distance V2 between the upper edge of a terminal end of sensor trace T5 (shown as H in FIG. 5) and the lower edge of a terminal end of offset sensor trace T4 (shown at I in FIG. 5B) is generally at least about 22 stitches; and the vertical distance V3 between the lower edge of a terminal end of offset trace T4 (shown at I in FIG. 5B) and an upper edge of sensor trace T6 (shown as J in FIG. 5B) is generally at least about 18 stitches.

FIG. 5C illustrates positioning of conductive signal transfer contacts C4, C5, C6 and common/ground contacts GC1, GC2 that communicate electrically with the respective traces and conductive bridges. The positioning of the conductive bridges is shown in the FIG. 4B image. Transfer contacts C4-C6, GC1, GC2 are positioned with center-to-center spacing of at least about 3 mm; transfer contact center-to-center spacing is often at least about 5 mm and, in some embodiments, center-to-center transfer contact spacing is about 1 cm. Neighboring transfer contacts are generally offset (vertically) with respect to one another, on center, by at least about 2 mm, often by at least about 5 mm, and in some embodiments, by about 1 cm or more.

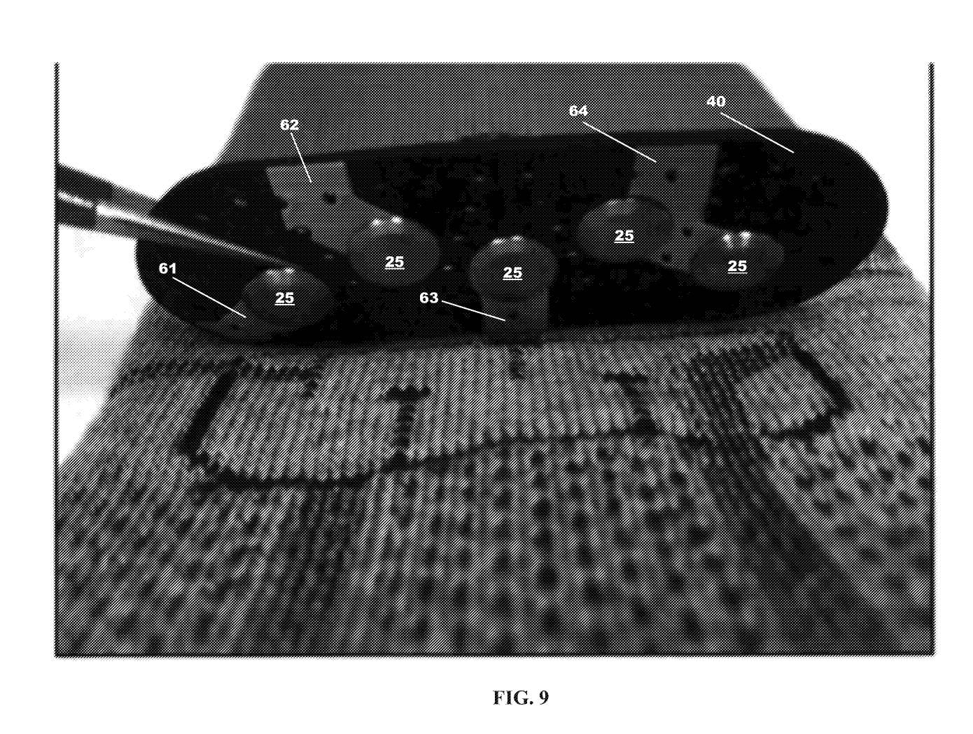

In some embodiments, as illustrated in FIGS. 6A and 6B, electrically conductive signal terminals may be provided as conductive terminal “buttons” 25 mounted through a non-conductive, flexible band 30 positioned and mounted on the sock to provide reliable contact between rearwardly-facing contact surfaces 26 and corresponding and underlying conductive signal traces and/or conductive bridges. Rear contact surfaces 26 of a plurality of conductive buttons 25 are exposed on an inner (underlying) surface 31 of mounting band 30, as shown in FIG. 6A, and externally facing terminal surfaces 27 project from an exterior surface 32 of mounting band 30, which is mounted on a sock or another garment, in the embodiment shown in FIG. 6B. In this embodiment, the conductive terminal buttons 25 are fabricated from an electrically conductive, non-corrosive iron-containing metallic material such as stainless steel.

The inwardly facing contact surfaces 26 of each conductive terminal button 25 in the embodiment shown in FIG. 6A comprise a generally flat or a slightly convex contact surface for establishing and maintaining reliable contact with an underlying conductive bridge or terminal area of a trace. A tapered or chamfered circumferential region 28 may be provided at the periphery of each conductive contact surface 26, as illustrated. The width of the tapered circumferential region 28 is generally less than 50% the diameter of the contact surface 26; in some embodiments, the width of the tapered circumferential region 28 is less than 30% the diameter of the contact surface 26; in yet other embodiments, the width of the tapered circumferential region 28 is less than 20% the diameter of the contact surface 26. The diameter of the contact surfaces 26 may range, in some embodiments, from approx. 2 mm to approx. 10 mm; in other embodiments, the diameter of contact surfaces 26 may range from approx. 3 mm to approx. 5 mm. While circular conductive terminals 25 and contact surfaces 26 are shown and discussed, it will be apparent that conductive terminals having alternative configurations may be used.

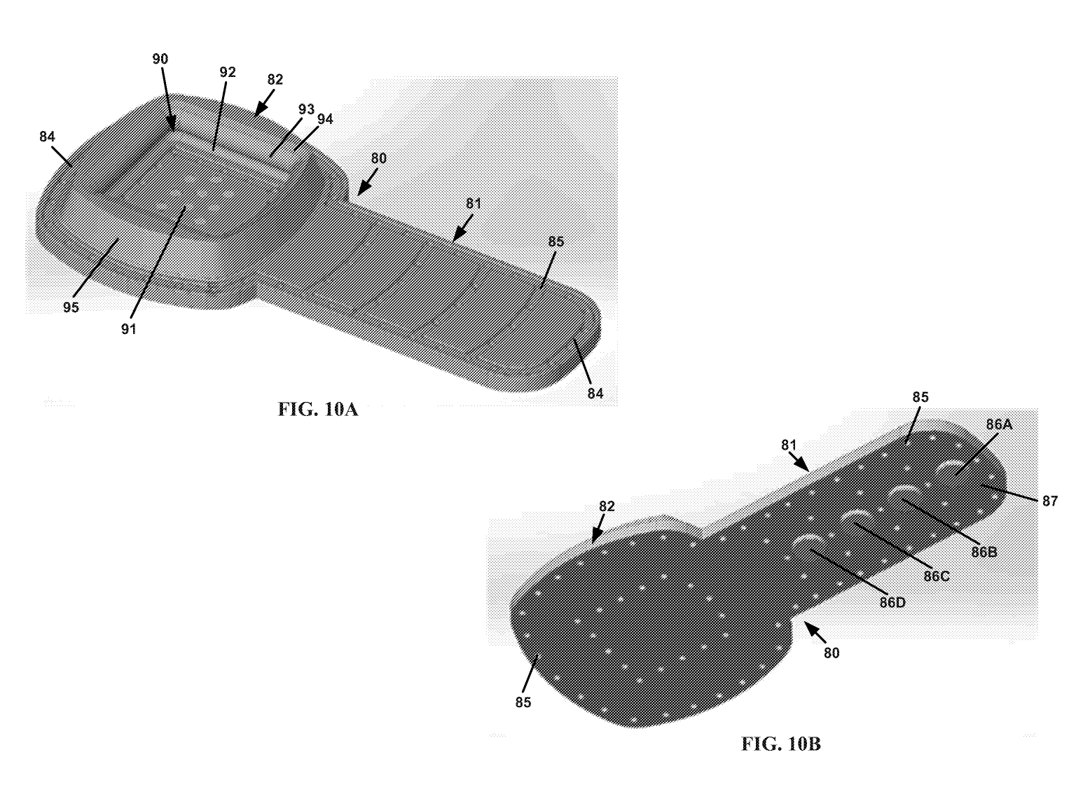

The external or outwardly-facing surfaces 27 of conductive signal transfer terminals 25, as shown in the exemplary embodiment illustrated in FIG. 6B, have contoured contact surfaces illustrated having a conical configuration. The location of the central region or apex of conical contact surfaces 27 may be elevated with respect to band 30 by a distance of about 2 to about 5 mm. The signal transfer and ground terminals have the same configuration in the illustrated embodiment, both using conductive terminal buttons 25. In this embodiment, the contoured contact surfaces 27 of conductive terminals 25 mate with correspondingly-configured recesses 36 provided in a curved DED 35, shown in FIG. 7. Contact recesses 36 provided in DED 35 may be fabricated wholly or partially from magnetic materials, so that mating of the metallic contoured contact surfaces 27 in corresponding recesses 36 of DED 35 is facilitated by attracting magnetic forces.

Mounting band 30, as illustrated in FIGS. 6A-7 is preferably fabricated from a durable, flexible, non-conductive material, such as a flexible plastic or rubbery material. Mounting band 30, as illustrated, has a generally oblong configuration and is attached/bonded/fixed/adhered to the underlying substrate (e.g., sock). An elevated rim 38 may be provided around the periphery of the band, as shown; the elevated rim may be provided around the entire periphery, as shown, or an elevated band may be provided around portion(s) of the periphery of the band.

In the illustrated embodiment, a peripheral groove 33 provided in exterior surface 32 has spaced bores or perforations 34 for stitching the mounting band to the substrate. Mounting band 30 additionally has grooves with spaced bores for attaching the mounting band to the underlying substrate provided at locations between neighboring conductive terminals 25. The mounting band perforation pattern is clearly shown in FIGS. 6B and 7; it will be recognized that perforations having different configurations, sizes and arrangements may be provided. This arrangement provides anchoring of each of the conductive terminals 25 to the substrate in the area of the conductive bridge or the terminal area of the trace, such as by stitching, and thereby facilitates stable positioning of the conductive terminal contact surfaces at, and in contact with, the underlying conductive bridge or the terminal trace area.

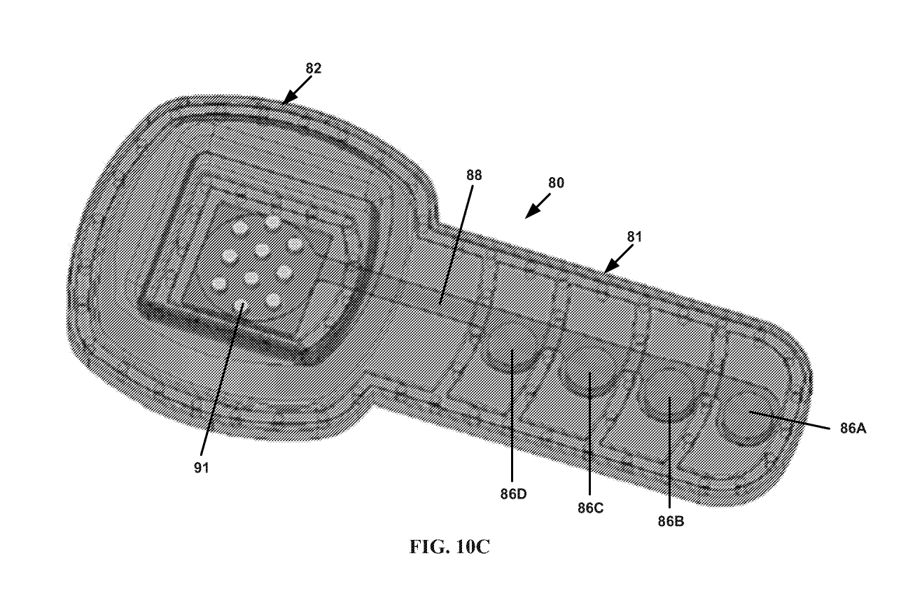

FIGS. 8A-8E illustrates a similar mounting band 40 having a plurality of bores for receipt of conductive terminals provided as cavities 46. In the embodiment illustrated in FIGS. 8A-8F, each of the receiving cavities 46 is aligned at a location offset from its neighbor(s), forming a staggered terminal arrangement, such as an “M” or “W” configuration. Each of the receiving cavities 46 is positioned in a thickened portion (“island”) 47 of the mounting band, with a peripheral groove 43, and intermediate grooves 45 provided between thickened islands 47. Grooves 43, 45 and perforations 44 provided in the grooves may provide additional band flexibility and facilitate bending of the band, and may also provide for attachment (such as by stitching) of the band 40 to an underlying substrate. A peripheral rim 48 may be provided extending around the periphery of band 40. The overall thickness of the mounting band 40 is generally from about 0.5 mm to about 4 mm; in some embodiments, the overall thickness of mounting band 40 is about 2 mm.

FIGS. 8E-8F illustrate an alternative embodiment in which conductive bridges are mountable to the mounting band to facilitate electrical contact between conductive traces or conductive bridges associated with the substrate and conductive terminals penetrating the mounting band. FIG. 8E illustrates conductive or partially conductive fittings 50, 51, 52, 53 that may be associated with the non-conductive band 40 and with conductive terminals mounted in the non-conductive band, providing conductive bridges associated with band 40 facilitating electrical connection between conductive traces and/or conductive bridges provided in an underlying substrate (such as a sock) and conductive terminals mounted in bores of the band. Conductive bridges associated with the band may be provided alternatively or in addition to conductive bridges associated with the trace terminals and/or substrate.

The conductive or partially conductive fittings 50, 51, 52, 53 illustrated in FIG. 8E may be fabricated from a variety of materials, including conductive thermoplastic elastomers incorporating a conductive filler. Thermoplastic elastomers (TPEs) comprising a conductive filler such as carbon black are suitable, including TPEs such as DRYFLEX C3. The individual fittings may include a conductive collar 55 sized and configured to be received within a cavity 46 of band 40, the collar 55 surrounding a bore 56 sized to receive a conductive terminal. The conductive fittings 50, 51, 52, 53 may also include one or more conductive extensions 57 projecting from collars 55 for a distance, as shown in FIG. 8E. For some applications, two (or more) collars 55 may be joined to one another and provided with a single conductive extension, as shown in conductive fitting 50. In some embodiments, conductive fittings 50 having joined collar portions may be provided in association with ground traces and ground terminals.

Conductive fittings 50, 51, 52, 53 may be associated with band 40 using various attachment systems, such as adhesives, fasteners, or the like. In some embodiments the band and conductive fitting combination may be fabricated and associated during processing, such as during a two shot manufacturing process. Using a two shot manufacturing technique, the band 30 may be formed from a suitable non-conductive material (e.g., a non-conductive TPE) in a first shot, and the fittings 50-53 may be formed, directly on the tab, as a second shot using a suitable conductive material (e.g., a conductive TPE). In some embodiments, the non-conductive band 40 and the conductive fittings 50-53 may comprise flexible materials having a durometer of less than about 100 on a Shore A scale. In some embodiments, the non-conductive tab 40 and conductive fittings 50-53 may comprise flexible materials characterized by different hardnesses. In some embodiments, the non-conductive band may comprise a material having a higher hardness on a Shore A scale than that of the conductive fittings. In some embodiments, the band may comprise a non-conductive TPE material having a hardness of about 80 Shore A; the conductive fittings may comprise a conductive TPE having a hardness of about 70 Shore A.

FIG. 8F illustrates a schematic bottom (inner) view of a non-conductive band 40 with conductive fittings 50, 51, 52, 53 installed or formed, as described above. Conductive extensions 57 extend from the receiving cavities 41 of band 40 and collars of conductive fittings 50-53 toward the periphery of band 40. When conductive terminals are inserted through the bores 56 of the conductive fittings, the band, with conductive fittings and conductive terminals in place, may be associated with (e.g., sewn or mounted or adhered or otherwise attached to) an underlying substrate to provide an electrical pathway from trace terminations located to contact the conductive fittings and/or conductive terminals.

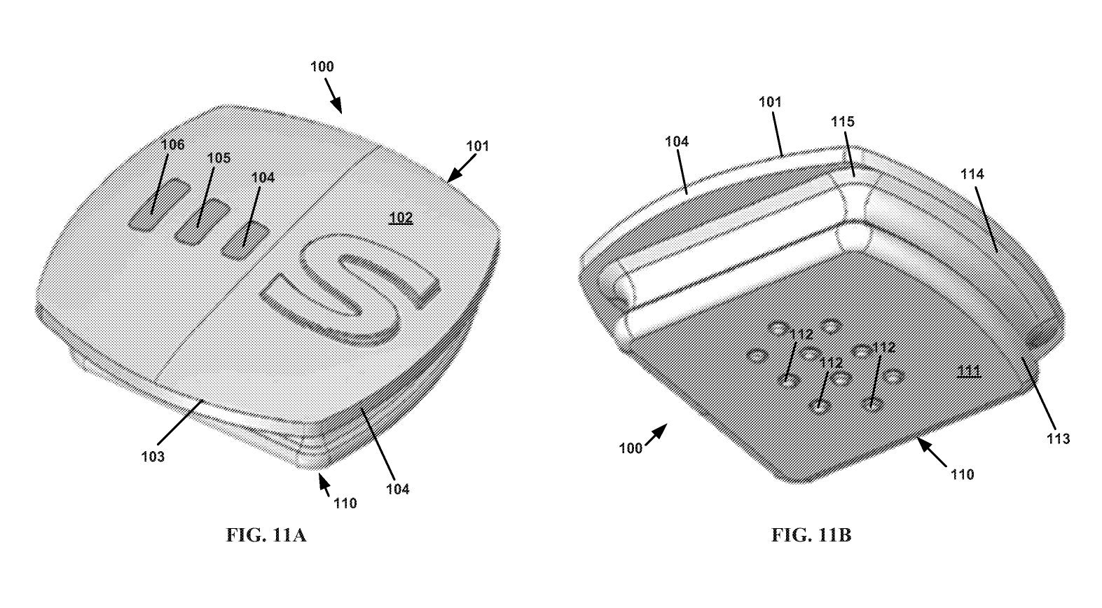

In another embodiment illustrated in FIG. 9, conductive pathways 61, 62, 63, 64 may be provided directly on a non-conductive band 40 in arrangements similar to those used for conductive fittings as described above. In this embodiment, conductive pathways 61, 62, 63, 64 may be provided between conductive terminals 25 and the peripheral edges of the non-conductive band 40. Conductive pathways 61-64 may be printed directly on the band using, for example, conductive TPE materials, conductive inks, or the like, facilitating electrical connection between the conductive terminals and the traces. In this embodiment, each conductive pathway 61-64 has a width that is at least 50%, in some embodiments at least 60%, and in some embodiments at least 80%, the largest surface dimension of the corresponding terminal. In the embodiment shown in FIG. 9, three sensor terminals are illustrated as the left-most terminals, with conductive pathways extending from underneath an edge of each of the terminals to a region near the edge of the band. Two common/ground terminals are illustrated as the right-most terminals, with conductive pathways provided between the two terminals and from one of the terminals to a region near the edge of the band.

The signal transfer terminals that connect to the sensor(s) in the sock are connectible to mating DED 35 in FIG. 7. The DED receives signals from each of the signal transfer terminals, and thus collects data from each of the sensors. The DED also interfaces with the ground terminal(s) and performs ground and auxiliary functions. In some embodiments, the DED may comprise data storage, processing and feedback capabilities, energy source(s), sensing components and functionality. In some embodiments, for example, a DED may incorporate an, a gyroscope, an orientation sensing component, a location sensing component, an inertial measurement unit, a temperature sensor, display capability, visual, audio and/or tactile indicating capabilities, and the like. It will be appreciated that many types and styles of DEDs may be provided for interfacing with and downloading signals and/or data from an underlying sock sensing device.

The signal transfer terminals for one sock (shown as “left” in FIG. 2) are provided in a “W” configuration, while the signal transfer terminals for the other sock (shown as “right” in FIG. 2) are provided in an “M” configuration. The spacing and configuration of terminals for each sock are thus identical, but inverted with respect to one another. This feature allows the same DED to be used with both the right and left sock, depending on the orientation of the DED—i.e., in one orientation, the signal receipt terminals on the DED have a “W” configuration; in another orientation rotated 180°, the signal receipt terminals on the DED have an “M” configuration. The signal transfer and receipt terminals are also coordinated, on right and left socks, and on the DEDs, so that the corresponding sensors on each sock, and the corresponding ground/common traces mate with the corresponding signal receipt terminals on the DED. This arrangement also allows the DED to distinguish which sock it's mounted on by sensing the orientation of the DED. It will be appreciated that many different arrangements of mating signal transfer and signal receipt terminals may be provided.

One or more signal transfer terminal(s), DED and/or mounting band may comprise a magnetic component, as previously mentioned. Magnetic field properties may be used to create terminal interfaces that can only connect in a predetermined orientation: in this way, the user is guided to properly connect the DED to the sensor system(s) associated with an underlying substrate. In addition, circuitry in the DED may provide the ability to automatically turn the data collection on and off, for example, based on the presence of the magnetic connection between the DED and the sensor system. It will be appreciated that many other types of mechanical and non-mechanical interfaces may be used to attach and detach the DED from the signal transfer terminals, and to transfer signals and/or data from the sensing system to the DED.

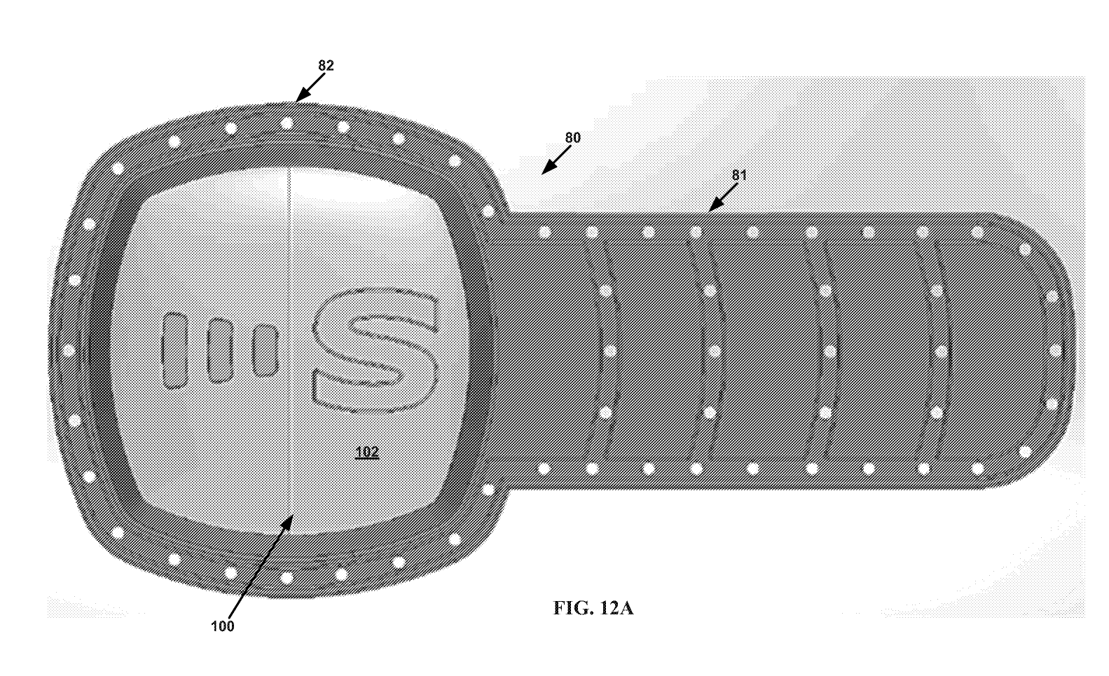

FIGS. 10A-13B illustrate another embodiment of a mounting tab and DED having alternative configurations for receiving signals from sensors and signal transfer terminals located on an underlying substrate. FIGS. 10A-10C show a mounting tab 80 having a band portion 81 and a DED receiving portion 82 including a DED receiving cavity 90 having a plurality of contacts 91 for mating with complementary contacts on a mating DED. Band and DED receiving portions 81, 82, respectively, may be associated with an underlying substrate (such as a garment) using a variety of mounting and attachment means, such as adhesives, material fusing, various types of fasteners, stitching, and the like. In the embodiment illustrated in FIGS. 10A-10C, grooves 84 having bores 85 are provided on an exterior (upper) surface along peripheral regions of band portion 81 and DED receiving portion 82 of mounting tab 80 to facilitate stitching of the mounting band to an underlying substrate. Additional grooves and bores may be provided traversing band portion 81 and along a bottom wall of DED receiving cavity 90, as shown.

A plurality of contacts (illustrated as contacts 86A, 86B, 86C, 86D) are provided penetrating and projecting from an interior (lower) surface 87 of band portion 81 of mounting tab 80. Contacts 86A-86D are configured and aligned for interfacing with and electrically contacting trace terminal portions or conductive bridges provided on an underlying substrate. The size, configuration, alignment and number of mounting tab contacts may vary depending on the size, configuration, alignment and number of trace terminations provided on a substrate.

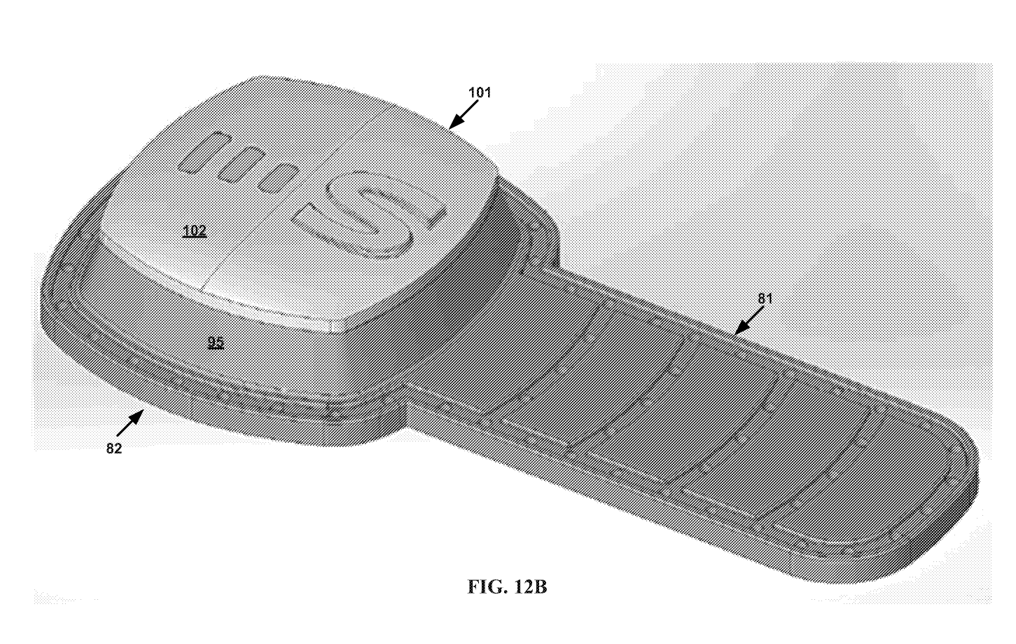

Contacts, illustrated as contacts 86A-D, are electrically connected to multiple electrically conductive pins 91 positioned in a contact interface region via electrical pathway 88, illustrated in FIG. 10C. The underside or interior surface of conductive pins 91 is visible in the view shown in FIG. 10C; the upper and DED interface surface of conductive pins 91 is visible in the view shown in FIG. 10A. The exposed interface surfaces of conductive pins 91 are located on an exposed surface of DED receipt cavity 90—on a lower, internal surface in the embodiment shown in FIG. 10A. The upper and side wall contours of DED receipt cavity 90 correspond generally to the outer contours of DED body, as shown in FIGS. 11A, 11B, to provide detachable yet stable mounting of DED body portion 110 within DED receipt cavity 90. Exterior surfaces of DED receipt cavity 90 may be provided as tapered external side walls 95.

In the specific embodiments illustrated in FIGS. 10A-11B, DED 100 comprises an exterior surface member 101 and an internal DED body 110 having an exterior configuration that mates with mounting tab DED receiving cavity 90. Exterior surface member 101 comprises an exterior surface 102 that may be smooth or contoured, and may have raised decorative or marketing indicators, system status indicators, or the like. Exterior surface member 101 has a perimeter wall 103 having a rounded polygonal configuration and a peripheral rim 104. Exterior surface member 101 may display optional indicators such as indicators 105, 106, 107 (e.g., LEDs) for communicating various system operational conditions, charge status, operational status, and the like. Exterior surface member 101 may carry additional or different user interface features, actuators, displays, decorative matter, and the like. Exterior surface member 101, as illustrated, has a perimeter larger than that of internal DED body 110.

Internal DED body 110, as shown in FIGS. 11A, 11B, comprises an internal surface 111 and a plurality of conductive pins 112 exposed on and/or projecting from internal surface 111. Conductive pins 112 may be provided as spring-loaded conductive pins, often referred to as pogo pins, to facilitate reliable contact with contacts 91 in the DED receiving cavity 90. Side walls of internal DED body 110 have a contoured configuration that is complementary to the contoured configuration of DED receipt cavity 90, facilitating convenient, stable and detachable positioning of DED 100 within DED receipt cavity 90. In the specific embodiment illustrated, internal DED body 110 comprises an internal rim 113, an intermediate groove 114 and an interface edge 115, each contoured surface being sized and configured for mating with complementary features of the DED receiving cavity 90, including internal channel 92, lip 93 and interface surface 94, respectively.

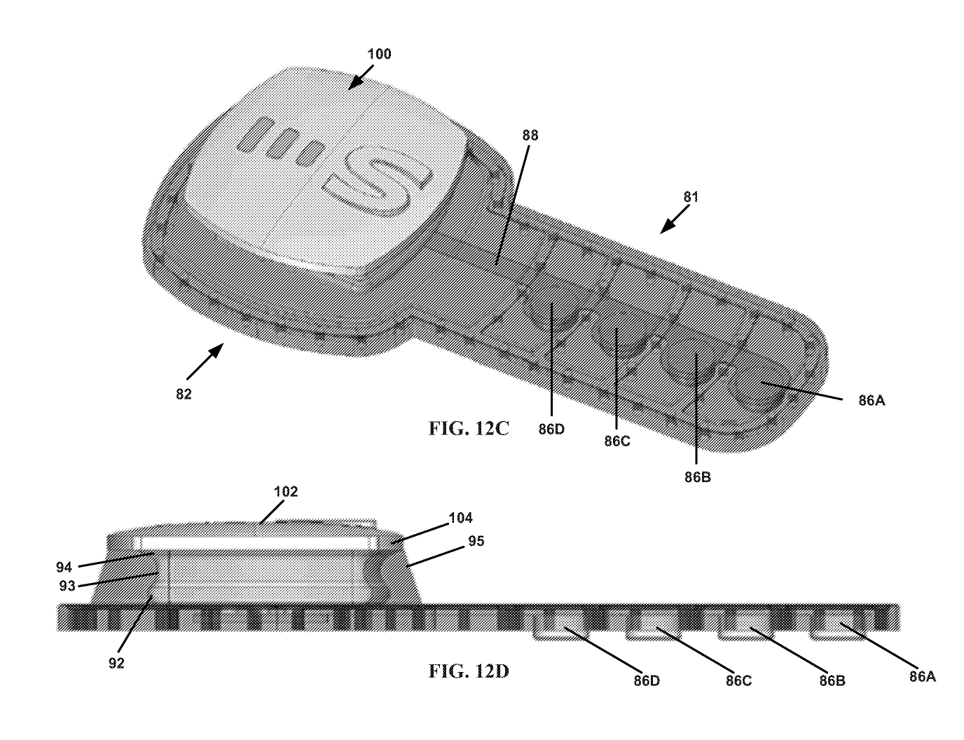

FIGS. 12A-12D show DED 100 mounted in mounting tab 80. Internal DED body 110 is enclosed within DED receipt cavity 90 in a substantially sealed manner. Tapered external side walls 95 of the DED receiving portion 82 and rim 104 are sized and configured to align and substantially seal DED receipt cavity 90. While DED 100, internal DED body 110 and DED receipt cavity 90 are illustrated having a generally square perimeter, it will be appreciated that other configurations may be used, including circular, oblong, other polygonal configurations, and other curved configurations.

Mounting tab 80 is generally constructed from a flexible, bendable non-conductive material such as a non-conductive, flexible thermoplastic elastomer (TPE), silicone, or the like. DED 100 is generally constructed from a harder, more rigid material, and may house electrical and electronic components such as one or more accelerometer(s); one or more gyroscope(s); one or more magnetometer(s); one or more 6-axis and/or 9-axis inertial measurement units IMU(s); data processing; data storage (e.g., flash memory); data communications (e.g., Bluetooth, ANT+, wi-fi; and/or Proprietary TX/RX protocols; energy source(s) (e.g., rechargeable battery/ies); antenna/e for wireless communications; and a plurality of analog sensor inputs (for pressure, temperature, humidity, and other sensor parameters).

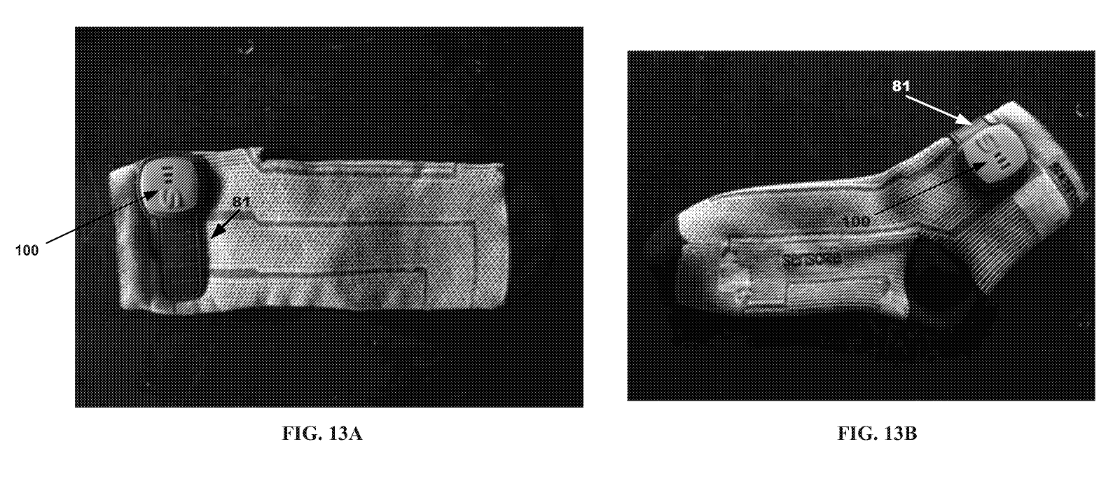

FIGS. 13A, 13B illustrate a mounting band 80 and DED 100 as illustrated in FIGS. 10A-12D mounted to a sock in an ankle region. The band portion of the mounting tab may be attached to the sock near an ankle region, traversing a front portion of the ankle region. The DED is positioned in a DED receiving cavity and is positioned laterally, on one side or the other, of the front or back side of the ankle region. It will be appreciated that this type of mounting band and DED may be used in association with other types of garments, including shirts, tunics, shorts, body suits, leotards, underwear, leggings, socks, footies, gloves, caps, bands such as wrist bands, leg bands, torso and back bands, brassieres and other types of substrates, including, for example, bands, bandages, and wound dressing materials, as well as with other types of accessories that contact a user's body surface (directly or indirectly) such as insoles, shoes, boots, belts, straps, and the like.

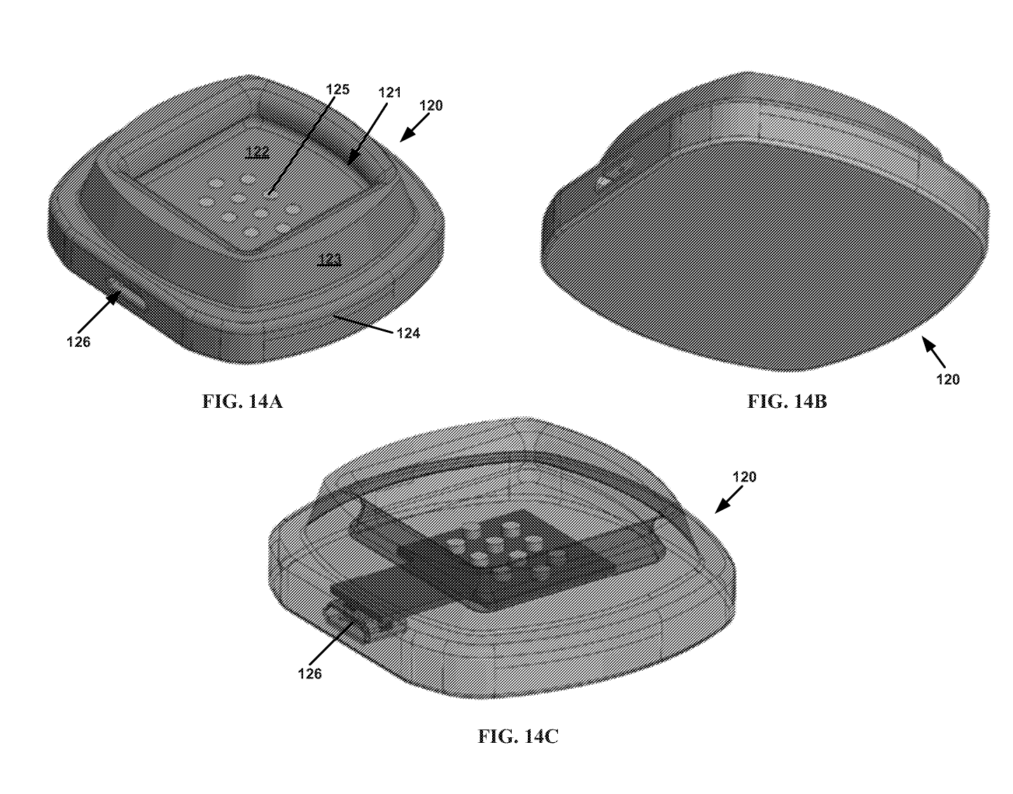

FIGS. 14A-14C show one embodiment of a charging station 120 for charging a DED component 100 having the configuration illustrated in FIGS. 11A-11B. The configuration of charging station 120, as illustrated, is similar to the configuration of DED receiving portion 82 of mounting tab 80. Exposed interface surfaces of conductive pins 125 are located on an exposed surface of DED receipt cavity 121, on a lower, internal cavity surface 122 in the embodiment shown in FIG. 14A. The upper and side wall contours of DED receipt cavity 121 correspond to the outer contours of DED body, as shown in FIGS. 11A, 11B, to provide detachable yet stable mounting of DED body portion 110 within DED receipt cavity 121 of charging station 120. Exterior surfaces of DED receipt cavity 121 may be provided as tapered external side walls 123. Charger base 124 provides stable positioning of the charging station 120 and locates electrical charging interface 126.

While specific examples of sensor systems and sensor system components, such as sensors, traces, conductive terminals and mounting bands are described with reference to a sock form factor, it will be appreciated that the features and components disclosed herein may be used with (and/or applied to) other types of wearable garments (e.g., underwear, t-shirts, trousers, tights, leggings, body suits, leotards, hats, gloves, bands, and the like), and many other types of substrates. Dedicated electronic devices having different configurations may be designed to interface with a variety of sensor systems embodied in different types of garments and other types of substrates. The type of sensor(s), garment(s), substrate(s), placement of sensor(s), DED, conductive terminal(s), and the like, may be varied for use in many different sensor system applications.

While the present invention has been described above with reference to the accompanying drawings in which specific embodiments are shown and explained, it is to be understood that persons skilled in the art may modify the embodiments described herein without departing from the spirit and broad scope of the invention. Accordingly, the descriptions provided above are considered as being illustrative and exemplary of specific structures, aspects and features within the broad scope of the present invention and not as limiting the scope of the invention. The various embodiments described herein may be combined to provide further embodiments. The described devices, systems and methods may omit some elements or acts, may add other elements or acts, or may combine the elements or execute the acts in a different order than that illustrated, to achieve various advantages of the disclosure. These and other changes may be made to the disclosure in light of the above detailed description.

In the present description, where used, the terms “about” and “consisting essentially of” mean±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives, unless otherwise expressly indicated. As used herein, the terms “include” and “have” and “comprise” are used synonymously, and those terms, and variants thereof, are intended to be construed as non-limiting. In general, in the following claims, the terms used should not be construed to limit the disclosure to the specific embodiments disclosed in the specification.

1. A sensor system comprising a non-electrically conductive substrate having at least one pressure sensor associated therewith, the at least one sensor being in electrical communication with at least one electrically conductive trace that terminates at or in proximity to a signal transfer terminal, and a dedicated electronic device (DED) having a signal receipt terminal that mates with each signal transfer terminal and a housing component, wherein the DED comprises a device selected from the group consisting of: an accelerometer; a gyroscope; an orientation sensing component; a location sensing component; an inertial measurement unit (IMU); a magnetometer; and a temperature sensor.

2. (canceled)

3. The sensor system of claim 1, wherein the DED housing component is detachably mountable to the substrate to provide mating of signal transfer terminals and signal receipt terminals of the DED.

4. The sensor system of claim 1, wherein each conductive trace terminates at a trace termination and additionally comprising at least one conductive bridge providing electrical contact between each trace termination and each signal receipt terminal, wherein each conductive bridge is elevated with respect to a surface of the non-electrically conductive substrate.

5. (canceled)

6. The sensor system of claim 4, wherein the at least one conductive bridge is fabricated from a material comprising a conductive thread or a conductive thermoplastic elastomer or a conductive ink or a conductive metallic material.

7. (canceled)

8. (canceled)

9. (canceled)

10. (canceled)

11. The sensor system of claim 1, wherein the at least one electrically conductive trace is fabricated from a material comprising a conductive thread or a conductive thermoplastic elastomer or a conductive ink or a conductive metallic material.

12. The sensor system of claim 1, wherein the non-electrically conductive substrate is a garment.

13. The sensor system of claim 12, wherein the garment is selected from the group consisting of: a shirt; underwear; leggings; a footie; a sock; a glove; a cap; a sleeve; a body band; a brassiere; shorts; pants; a body suit; and a leotard.

14. The sensor system of any of claim 1, wherein the non-electrically conductive substrate is associated with an insole; a shoe; a boot; a belt; a strap; a wrap; a bandage; a wound dressing; a sheet; a pad; a cushion; or sporting equipment.

15. (canceled)

16. The sensor system of claim 1, wherein the at least one sensor provides data relating to the location and magnitude of impact forces exerted against the sensors.

17. The sensor system of claim 16, wherein the non-electrically conductive substrate is a sock.

18. A sensor system comprising: a non-electrically conductive substrate having a plurality of sensors associated therewith, each of the sensors being in electrical communication with at least one electrically conductive trace that terminates at or in proximity to a trace termination; a dedicated electronic device (DED) receiving portion associated with the substrate having a plurality of signal receipt contacts configured and aligned to interface with the trace terminations, the DED receiving portion having a plurality of signal transfer contacts communicating with the signal receipt contacts and arranged in a DED receiving cavity; and a dedicated electronic device (DED) body having signal receipt terminals that mate with the signal transfer contacts in the DED receiving cavity, wherein the DED body is sized and configured to be detachably received in the DED receiving cavity.

19. The sensor system of claim 18, comprising at least one sensor selected from the following: a conductive electrode; a conductive e-textile sensor; a resistive e-textile sensor; a pressure or force sensor; a temperature sensor; a galvanic skin response sensor; a moisture sensor; a heart rate sensor; a respiration sensor; an electromyography sensor; a blood gas content sensor; a skin conductivity sensor; an accelerometer; a gyroscope; and a location or position sensor.

20. The sensor system of claim 18, wherein the DED body additionally comprises a device selected from the group consisting of: an accelerometer; a gyroscope; an orientation sensing component; a location sensing component; an inertial measurement unit (IMU); a magnetometer; and a temperature sensor.

21. (canceled)

22. The sensor system of claim 18, wherein the DED body has an exterior surface member displaying visual indicators.

23. The sensor system of claim 18, wherein the DED body has an exterior surface member displaying user interface features.

24. (canceled)

25. (canceled)

26. (canceled)

27. (canceled)

28. (canceled)

29. (canceled)

30. (canceled)

31. (canceled)

32. (canceled)

33. (canceled)

34. The sensor system of claim 1, wherein the at least one pressure sensor is sandwiched between substrate layers.

35. The sensor system of claim 1, wherein the at least one pressure sensor is sandwiched between film layers.

36. The sensor system of claim 18, wherein the at least one pressure sensor is sandwiched between film layers.

37. The sensor system of claim 18, additionally comprising an exterior surface member associated with the DED body, wherein the exterior surface member has a perimeter larger than that of the DED body.

38. The sensor system of claim 37, wherein the DED body comprises an internal rim and an intermediate groove.
